(19) United States
(12) Patent Application Publication
(10) Pub. No.: US 2015/0056249 A1
(43) Pub. Date: Feb. 26, 2015

(54) INFLUENZA VACCINES

(71) Applicant: GLAXOSMITHKLINE BIOLOGICALS, SA, Rixensart (BE)

(72) Inventors: Bruce Lamont Innis, King of Prussia, PA (US); Sumita Roy-Ghanta, King of Prussia, PA (US)

(21) Appl. No.: 14/386,096

(22) PCT Filed: Mar. 13, 2013
(86) PCT No.: PCT/EP2013/055104

§ 371 Date: Sep. 18, 2014

(30) Foreign Application Priority Data
Mar. 23, 2012 (GB) 1205189.2

(51) Int. Cl. A61K 39/145

(52) U.S. Cl. CPC A61K 39/145
USPC 424/209.1

(57) ABSTRACT

The present invention relates to influenza vaccine compositions and vaccination schemes for immunizing against influenza disease, in particular it relates to immunogenic compositions comprising an antigen or a antigenic preparation from a first influenza virus strain and an oil-in-water emulsion adjuvant for use in inducing a immune response against at least one second influenza virus strain wherein said second influenza virus strain is from a different type or from a different subtype than said first influenza virus strain.

### TECHNICAL FIELD

The present invention relates to influenza vaccine compositions and vaccination schemes for immunising against influenza disease, in particular for inducing cross-protective immune responses against influenza virus strains which are not included within the vaccine compositions, and maintaining those responses in a persistent way, preferably for at least a few months.

### BACKGROUND TO INVENTION

Influenza viruses are one of the most ubiquitous viruses present in the world, affecting both humans and livestock. Influenza results in an economic burden, morbidity and even mortality, which are significant. There are three types of influenza viruses: A, B and C.

The influenza virus is an enveloped virus which consists basically of an internal nucleocapsid or core of ribonucleic acid (RNA) associated with nucleoprotein, surrounded by a viral envelope with a lipid bilayer structure and external glycoproteins. The inner layer of the viral envelope is composed predominantly of matrix proteins and the outer layer mostly of host-derived lipid material. Influenza virus comprises two surface antigens, glycoproteins neuraminidase (NA) and haemagglutinin (HA), which appear as spikes at the surface of the particles. It is these surface proteins, particularly HA that determine the antigenic specificity of the influenza subtypes.

Virus strains are classified according to host species of origin, geographic site and year of isolation, serial number, and, for influenza A, by serological properties of subtypes of HA and NA. 16 HA subtypes (H1-H16) and nine NA subtypes (N1-N9) have been identified for influenza A viruses [Webster R G et al. Evolution and ecology of influenza A viruses. Microbiol. Rev. 1992; 56:152-179; Fouchier R A et al. Characterization of a Novel Influenza A Virus Hemagglutinin Subtype (H16) Obtained from Black-Headed Gulls. J. Virol. 2005; 79:2814-2822). Viruses of all HA and NA subtypes have been recovered from aquatic birds, but only three HA subtypes (H1, H2, and H3) and two NA subtypes (N1 and N2) have established stable lineages in the human population since 1918. Only one subtype of HA and one of NA are recognised for influenza B viruses.

Influenza A-type viruses evolve and undergo antigenic variability continuously [Wiley D, Skehel J. The structure and the function of the hemagglutinin membrane glycoprotein of influenza virus. Ann. Rev. Biochem. 1987; 56:365-394]. A lack of effective proofreading by the viral RNA polymerase leads to a high rate of transcription errors that can result in amino-acid substitutions in surface glycoproteins. This is termed “antigenic drift”. The segmented viral genome allows for a second type of antigenic variation. If two influenza viruses simultaneously infect a host cell, genetic reassortment, called “antigenic shift” may generate a novel virus with new surface or internal proteins. Influenza virus strain resulting from an antigenic shift, in particular, may cause a pandemic.

Vaccination plays a critical role in controlling influenza epidemics. Currently available influenza vaccines are either inactivated or live attenuated influenza vaccines. Inactivated flu vaccines are composed of three possible forms of antigen preparation: inactivated whole virus, sub-virions where purified virus particles are disrupted with detergents or other reagents to solubilise the lipid envelope (so-called “split” vaccine) or purified HA and NA (subunit vaccine). These inactivated vaccines are usually given intramuscularly (i.m.), subcutaneously (s.c), or intranasally (i.n.).

Influenza vaccines for interpandemic use (also termed seasonal), of all kinds, are usually trivalent vaccines. They generally contain antigens derived from two influenza A-type virus strains and one influenza B-type virus strain. A standard 0.5 ml injectable dose in most cases contains (at least) 15 μg of HA from each strain, as measured by single radial immunodiffusion (SRD) (J. M. Wood et al.: An improved single radial immunodiffusion technique for the assay of influenza haemagglutinin antigen: adaptation for potency determination of inactivated whole virus and subunit vaccines. J. Biol. Stand. 5 (1977) 237-247; J. M. Wood et al., International collaborative study of single radial diffusion and immunoelectrophoresis techniques for the assay of haemagglutinin antigen of influenza virus. J. Biol. Stand. 9 (1981) 317-330). Usually, those vaccines are unadjuvanted.

New vaccines with a cross-protection potential that could be used as pre-pandemic or stockpiling vaccines to prime an immunologically naive population against a pandemic strain before or upon declaration of a pandemic have been recently developed. Such vaccines are formulated with potent adjuvants for enhancing immune responses to subvirion antigens. For example, WO2008/009309 or Leroux-Roels et al. (PLos ONE, 2008. 3(2): 1-5) disclose vaccines comprising an influenza antigen associated with a pandemic in combination with an adjuvant comprising an oil-in-water emulsion. In particular, it was observed that vaccination with an oil-in-water adjuvanted immunogenic composition comprising a H5N1 influenza virus strain of clade 1 produced cross-reactivity against an H5N1 influenza virus strain of clade 2. Another study has reported the administration of a pandemic vaccine adjuvanted with an oil-in-water emulsion followed by the administration of the next seasonal trivalent vaccine (Gilca et al., Vaccine. 2011, 30(1): 35-41).

Another study has reported that two doses of an H5N3 influenza vaccine adjuvanted with MF59 was boosting immunity to influenza H5N1 in a primed population (Stephenson et al., Vaccine 2003, 21, 1687-1693). A further study has reported cross-reactive antibody responses to H5N1 viruses obtained after three doses of a particular oil-in-water emulsion adjuvanted influenza H5N3 vaccine (Stephenson et al., J. Infect. Diseases 2005, 191, 1210-1215).

However, there is still a need for vaccine compositions and vaccination strategies capable of providing broader cross-protection, in particular cross-protection with respect to influenza viruses of different subtypes, and to influenza viruses of different types, possibly to multiple different strains, as well as a broader cross-protection which persists over time.

### SUMMARY OF THE INVENTION

In a first aspect of the invention, there is provided an immunogenic composition comprising an antigen or an antigenic preparation from a first influenza virus strain and an oil-in-water emulsion adjuvant for use in inducing an immune response against at least one second influenza virus strain which is from a different type or from a different subtype than said first influenza virus strain.

In a second aspect of the invention, there is provided a second immunogenic composition comprising an antigen or an antigenic preparation from at least one influenza virus strain for use according to a one dose scheme in a paediatric subject which has previously been vaccinated with a first immunogenic composition comprising an antigen or an antigenic preparation from at least one influenza virus strain and oil-in-water emulsion adjuvant.

In a third aspect, there is provided an immunogenic composition comprising an antigen or an antigenic preparation from a first influenza virus strain and an oil-in-water emulsion adjuvant for use in the treatment or prevention of disease caused by a second influenza virus strain wherein said second influenza virus strain is from a different subtype or a different type than said first influenza virus strain.

In a fourth aspect, there is provided a method of prevention and/or treatment against influenza disease, wherein a first immunogenic composition comprising an antigen or an antigenic preparation from at least one influenza virus strain together with an oil-in-water emulsion adjuvant is first administered and a second immunogenic composition comprising an antigen or an antigenic preparation from at least one influenza virus strain is administered.

### BRIEF DESCRIPTION OF THE DRAWINGS

FIG. 1. H1N1 priming in a preclinical prime-boost vaccination mouse model. Priming with Pandemrix™ followed by Fluarix™ boost gave higher HI titers against A/H3N2/Victoria and B/Brisbane (and A/H1N1/California) compared to one administration of Fluarix™. See Example 3. N=12 mice per condition. GMT=geometric mean titer.

### DETAILED DESCRIPTION

The present inventors have observed that a population of subjects vaccinated with an immunogenic composition comprising an influenza antigen from a first influenza virus strain, together with an oil-in-water emulsion adjuvant displayed an improved immune response in response to vaccination with a second immunogenic composition comprising an influenza antigen from the same influenza virus strain, as compared to that obtained in a population of subjects which was only vaccinated with the second immunogenic composition. In addition, the inventors discovered that such a prior vaccination allowed to achieve an improved immune response in response to vaccination with a second immunogenic composition comprising an influenza antigen from a second influenza virus strain which is of a different subtype or of a different type, as compared to that obtained in a population of subjects which was only vaccinated with the second immunogenic composition. This indicates that influenza formulations adjuvanted with an oil-in-water emulsion adjuvant can advantageously be used to induce a cross-reactive immune response, i.e. detectable immunity (humoral and/or cellular) against a variant strain or against a range of related strains. They can also advantageously be used to induce a cross-priming strategy, i.e. induce “primed” immunological memory facilitating response upon re-vaccination (one-dose) with the same influenza virus strain and/or different strains.

In particular, the inventors surprisingly observed that a prior vaccination with an immunogenic composition comprising an A-type influenza virus strain together with an oil-in-water emulsion adjuvant resulted in improved immune responses in response to vaccination with an immunogenic composition comprising a B-type influenza virus strain, indicating that the cross-priming strategy is not limited to closely related influenza virus strains.

Accordingly, it is an object of the present invention to provide a method of prevention and/or treatment against influenza disease, wherein a first immunogenic composition comprising an antigen or an antigenic preparation from at least one influenza virus strain together with an oil-in-water emulsion adjuvant is first administered, suitably according to a one dose-scheme, and a second immunogenic composition comprising an antigen or an antigenic preparation from an influenza virus strain is administered thereafter, suitably according to a one dose-scheme. In one embodiment, the at least influenza virus strains of the first immunogenic composition and of the second immunogenic composition are of a different type or a different subtype. Suitably the first immunogenic composition is administered at the declaration of a pandemic and the second immunogenic composition is administered later. Alternatively the administration of the first immunogenic composition is part of a pre-pandemic strategy and is made before the declaration of a pandemic, as a priming strategy, thus allowing the immune system to be primed, with the administration of the further/boosting immunogenic composition made subsequently. Typically the second immunogenic composition is administered at least 4 months after the first immunogenic composition, suitably 6 or 8 to 14 months after, suitably at around 10 to 12 months after, for example 12 months, or even longer. Suitably the administration of the second immunogenic composition one year later or even more than one year later is capable of boosting antibody and/or cellular immune responses. This is especially important as further waves of infection may occur several months after the first outbreak of a pandemic. As needed, the administration of the second immunogenic composition may be made more than once, e.g. twice. In one embodiment, there is provided a method of prevention and/or treatment against influenza disease, wherein a first immunogenic composition comprising an antigen or an antigenic preparation from at least one influenza virus strain together with an oil-in-water emulsion adjuvant is first administered, and a second immunogenic composition comprising an antigen or an antigenic preparation from an influenza virus strain is administered at least 6 months later, such as one year later.

Surprisingly, the improved immune responses which were achieved when the population of subjects was first vaccinated with a first immunogenic composition comprising an influenza antigen from a first influenza virus strain together with an oil-in-water emulsion adjuvant were observed after one dose only of the first immunogenic composition and one dose only of the second immunogenic composition comprising an influenza antigen derived from a second influenza virus strain.

The inventors additionally observed that the immunogenic compositions for use in the present invention are able not only to induce but also to maintain significant levels of immune responses over time against not only the influenza virus strain present in the first immunogenic composition, but also against influenza virus strains of a different type or a different subtype. Therefore, the immunogenic compositions for use according to the invention are capable of ensuring a persistent immune response against influenza disease caused by influenza virus strains which are (i) identical to, (ii) of a type or (iii) of a subtype different from, the strain included in the first immunogenic composition. In particular, by persistence it is meant an antibody response which is capable of meeting regulatory criteria after at least three months, suitably after at least 6 months, more suitably after at least 12 months, after the vaccination. In particular, the claimed composition for use according to the invention is able to induce protective levels of antibodies as measured by the protection rate (see Table 1) in >50%, suitably in >60% of individuals >70% of individuals, suitably in >80% of individuals or suitably in >90% of individuals for the influenza virus strain present in the vaccine, after at least three months. In a specific aspect, protective levels of antibodies of >90% are obtained at least 6 months post-vaccination against the influenza virus strain of the vaccine composition.

Accordingly, it is also an object of the present invention to provide influenza immunogenic compositions, such as vaccines, and vaccinations schemes for immunizing against influenza disease, in particular for inducing cross-protective immune responses against influenza virus strains which are not included within the immunogenic compositions, and maintaining those responses in a persistent way, suitably for at least a few months.

Influenza Viral Strains and Antigens

In one embodiment, an influenza virus or antigenic preparation thereof for use according to the present invention may be a split influenza virus or split virus antigenic preparation thereof. In an alternative embodiment the influenza preparation may contain another type of inactivated influenza antigen, such as inactivated whole virus or recombinant and/or purified HA and NA (subunit vaccine), or an influenza virosome. In a still further embodiment, the influenza virus may be a live attenuated influenza preparation.

A split influenza virus or split virus antigenic preparation thereof for use according to the present invention is suitably an inactivated virus preparation where virus particles are disrupted with detergents or other reagents to solubilise the lipid envelope. Split virus or split virus antigenic preparations thereof are suitably prepared by fragmentation of whole influenza virus, either infectious or inactivated, with solubilising concentrations of organic solvents or detergents and subsequent removal of all or the majority of the solubilising agent and some or most of the viral lipid material. By split virus antigenic preparation thereof is meant a split virus preparation which may have undergone some degree of purification compared to the split virus whilst retaining most of the antigenic properties of the split virus components. For example, when produced in eggs, the split virus may be depleted from egg-contaminating proteins, or when produced in cell culture, the split virus may be depleted from host cell contaminants. A split virus antigenic preparation may comprise split virus antigenic components of more than one viral strain. Vaccines containing split virus (called ‘influenza split vaccine’) or split virus antigenic preparations generally contain residual matrix protein and nucleoprotein and sometimes lipid, as well as the membrane envelope proteins. Such split virus vaccines will usually contain most or all of the virus structural proteins although not necessarily in the same proportions as they occur in the whole virus.

Alternatively, the influenza virus may be in the form of a whole virus vaccine. This may prove to be an advantage over a split virus vaccine for a pandemic situation as it avoids the uncertainty over whether a split virus vaccine can be successfully produced for a new strain of influenza virus. For some strains the conventional detergents used for producing the split virus can damage the virus and render it unusable. In addition to the greater degree of certainty with a whole virus approach, there is also a greater vaccine production capacity than for split virus since considerable amounts of antigen are lost during additional purification steps necessary for preparing a suitable split vaccine.

In another embodiment, the influenza virus preparation is in the form of a purified sub-unit influenza vaccine. Sub-unit influenza vaccines generally contain the two major envelope proteins, HA and NA, and may have an additional advantage over whole virion vaccines as they are generally less reactogenic, particularly in young vaccinees. Sub-unit vaccines can produced either recombinantly or purified from disrupted viral particles.

In another embodiment, the influenza virus preparation is in the form of a virosome. Virosomes are spherical, unilamellar vesicles which retain the functional viral envelope glycoproteins HA and NA in authentic conformation, intercalated in the virosomes' phospholipids bilayer membrane.

Said influenza virus or antigenic preparation thereof may be egg-derived or cell-culture derived. They may also be produced in other systems such as insect cells, plants, yeast or bacteria and/or be recombinantly produced.

For example, the influenza virus antigen or antigenic preparations thereof according to the invention may be derived from the conventional embryonated egg method, by growing influenza virus in eggs and purifying the harvested allantoic fluid. Eggs can be accumulated in large numbers at short notice. Alternatively, they may be derived from any of the new generation methods using tissue culture to grow the virus or express recombinant influenza virus surface antigens. Suitable cell substrates for growing the virus include for example dog kidney cells such as MDCK or cells from a clone of MDCK, MDCK-like cells, monkey kidney cells such as AGMK cells including Vero cells, suitable pig cell lines, or any other mammalian cell type suitable for the production of influenza virus for vaccine purposes. Suitable cell substrates also include human cells e.g. MRC-5 or Per-C6 cells. Suitable cell substrates are not limited to cell lines; for example primary cells such as chicken embryo fibroblasts and avian cell lines, such as EB66 cells, are also included.

The influenza virus antigen or antigenic preparation thereof may be produced by any of a number of commercially applicable processes, for example the split flu process described in patent no. DD 300 833 and DD 211 444, incorporated herein by reference. Traditionally split flu was produced using a solvent/detergent treatment, such as tri-/7-butyl phosphate, or diethylether in combination with Tween™ (known as “Tween-ether” splitting) and this process is still used in some production facilities. Other splitting agents now employed include detergents or proteolytic enzymes or bile salts, for example sodium deoxycholate as described in patent no. DD 155 875, incorporated herein by reference. Detergents that can be used as splitting agents include cationic detergents e.g. cetyl trimethyl ammonium bromide (CTAB), other ionic detergents e.g. laurylsulfate, taurodeoxycholate, or non-ionic detergents such as the ones described above including Triton X-100 (for example in a process described in Una et al, 2000, Biologicals 28, 95-103) and Triton N-101, or combinations of any two or more detergents.

The preparation process for a split vaccine may include a number of different filtration and/or other separation steps such as ultracentrifugation, ultrafiltration, zonal centrifugation and chromatography (e.g. ion exchange) steps in a variety of combinations, and optionally an inactivation step eg with heat, formaldehyde or β-propiolactone or U.V. which may be carried out before or after splitting. The splitting process may be carried out as a batch, continuous or semi-continuous process. A suitable splitting and purification process for a split immunogenic composition is described in WO 02/097072.

Suitable split flu vaccine antigen preparations according to the invention comprise a residual amount of Tween 80 and/or Triton X-100 remaining from the production process, although these may be added or their concentrations adjusted after preparation of the split antigen. Suitable ranges for the final concentrations of these non-ionic surfactants in the vaccine dose are:

Tween 80: 0.01 to 1%, suitably about 0.1% (v/v)

Triton X-100: 0.001 to 0.1 (% w/v), suitably 0.005 to 0.02% (w/v).

In a specific embodiment, the final concentration for Tween 80 ranges from 0.045%-0.09% w/v. In another specific embodiment, the antigen is provided as a 2-fold concentrated mixture, which has a Tween 80 concentration ranging from 0.045%-0.2% (w/v) and has to be diluted two times upon final formulation with the adjuvanted (or the buffer in the control formulation).

In another specific embodiment, the final concentration for Triton X-100 ranges from 0.005%-0.017% w/v. In another specific embodiment, the antigen is provided as a 2 fold concentrated mixture, which has a Triton X-100 concentration ranging from 0.005%-0.034% (w/v) and has to be diluted two times upon final formulation with the adjuvanted (or the buffer in the control formulation).

In one embodiment, the influenza preparation according to the invention is prepared in the presence of low level of preservative in particular thiomersal, or suitably in the absence of thiomersal.

As described earlier, influenza viruses can be classified into 3 types: A, B and C. Therefore, in the sense of the present invention, the terms “influenza type” are to be understood as A-type, B-type or C-type.

A-type influenza viruses can be further classified into different subtypes, based on their HA (16 subtypes, H1 to H16) and NA proteins (9 subtypes, N1 to N9), while B-type influenza viruses are known to be made of only one HA and one NA subtype. Accordingly, in the sense of the present invention, the term “influenza subtypes” is to be understood as A-type influenza virus strains having a given H subtype and a given N subtype, and the terms “different subtype” refer to influenza virus strains which do not share the same H subtype and/or the same N subtype.

In a one embodiment, the immunogenic compositions for use according to the invention comprise an antigen or an antigenic preparation from a first influenza virus strain and are used to induce an immune response against at least one second influenza virus strain having an H (HA) subtype different from the H (HA subtype) of the first influenza virus strain.

In a specific embodiment, the immunogenic compositions for use according to the invention comprise an antigen or an antigenic preparation from a first influenza virus strain and are used to induce an immune response against at least one second influenza virus strain having the same N (NA) subtype, but an H (HA) subtype different from the H (HA subtype) of the first influenza virus strain.

As described earlier, Influenza A viruses evolve and undergo antigenic variability continuously. A lack of effective proofreading by the viral RNA polymerase leads to a high rate of transcription errors that can result in amino-acid substitutions in surface glycoproteins, such as HA and NA proteins. This is termed “antigenic drift”. The segmented viral genome allows for a second type of antigenic variation. If two influenza viruses simultaneously infect a host cell, genetic reassortment, called “antigenic shift” may generate a novel virus with new surface or internal proteins. These antigenic changes, both ‘drifts’ and ‘shifts’ are unpredictable and may have a dramatic impact from an immunological point of view as they eventually lead to the emergence of new influenza virus strains and that enable the virus to escape the immune system causing the well known, almost annual, epidemics. Both of these genetic modifications have caused new viral variants responsible for pandemic in humans.

Accordingly, in the sense of the present invention, the term “variant strains” are to be understood as strains which are not identical, but underwent either an antigenic drift or an antigenic shift with respect to a reference strain.

The immunogenic compositions comprising an oil-in-water emulsion adjuvant for use in the invention may comprise an influenza antigen from any type (A-type, B-type, C-type) and any subtype (H1 to H16 and N1 to N9) of influenza viruses. Suitably, the influenza virus to be included in immunogenic compositions for use according to the invention is from a pandemic strain. By pandemic strain, it is meant a new influenza virus against which the large majority of the human population has no immunity. Throughout the document it will be referred to a pandemic strain as an influenza virus strain being associated or susceptible to be associated with an outbreak of influenza disease, such as pandemic Influenza A-type virus strains. Suitable pandemic strains are, but not limited to: H5N1, H9N2, H7N7, H2N2, H7N1 and H1N1. Others suitable pandemic strains in human are H7N3 (2 cases reported in Canada), H10N7 (2 cases reported in Egypt) and H5N2 (1 case reported in Japan). Alternatively, the influenza virus to be included in immunogenic compositions comprising an oil-in-water emulsion adjuvant for use according to the invention may be from a classical strain, i.e. a non-pandemic strain.

In one embodiment, the immunogenic composition for use according to the invention comprises an A-type influenza virus, such as H1, e.g. H1N1, H2, H5, e.g. H5N1, H7 or H9 and is used for inducing an immune response against at least one influenza virus of a different subtype, e.g. H3. In an alternative embodiment, the immunogenic composition for use according to the invention comprises an A-type influenza virus, such as H1, e.g. H1N1, H2, H5, e.g. H5N1, H7 or H9 and is used for inducing an immune response against at least one B-type influenza virus.

In one embodiment, the immunogenic oil-in-water emulsion adjuvanted composition for use according to the invention is monovalent, i.e. only comprises one influenza virus strain. In a specific embodiment, the monovalent immunogenic oil-in-water emulsion adjuvanted composition for use according to the invention comprises a pandemic influenza virus train or a strain having the potential to be associated with a pandemic. In alternative embodiments, the immunogenic oil-in-water emulsion adjuvanted composition for use according to the invention is multivalent, i.e. comprises multiple influenza virus strain. For example, the composition is suitably, bivalent, trivalent, or quadrivalent.

In one embodiment, the immunogenic oil-in-water emulsion adjuvanted composition for use according to the invention is used for inducing an immune response against multiple influenza virus strains, optionally including multiple strains from a subtype or a type different from the influenza virus strain(s) included in the immunogenic oil-in-water emulsion adjuvanted composition.

In a specific embodiment, the immunogenic oil-in-water emulsion adjuvanted composition for use according to the invention is used for inducing an immune response against one, two, three or all, of: an A/H1N1 strain, an A/H3N2 strain, a B strain of the Yagamata lineage and a B strain of the Victoria lineage.

In one embodiment, the influenza virus or antigenic preparation and the oil-in-water emulsion adjuvant for use according to the invention are contained in the same container. It is referred to as ‘one vial approach’. In another embodiment, the influenza virus or antigenic preparation and the oil-in-water emulsion adjuvant for use according to the invention is a 2 component vaccine, i.e. the antigenic preparation and the adjuvant are present in different containers, for mixture prior to the administration to the subject.

Oil-in-Water Emulsion Adjuvant

The adjuvant composition of the invention contains an oil-in-water emulsion adjuvant, suitably said emulsion comprises a metabolisable oil in an amount of 0.5% to 20% of the total volume, and having oil droplets of which at least 70% by intensity have diameters of less than 1 μm.

The meaning of the term metabolisable oil is well known in the art. Metabolisable can be defined as ‘being capable of being transformed by metabolism’ (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts, seeds, and grains are common sources of vegetable oils. Synthetic oils are also part of this invention and can include commercially available oils such as NEOBEE® and others. A particularly suitable metabolisable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly suitable oil for use in this invention. Squalene is a metabolisable oil by virtue of the fact that it is an intermediate in the biosynthesis of cholesterol (Merck index, 10th Edition, entry no. 8619).

Oil in water emulsions per se are well known in the art, and have been suggested to be useful as adjuvant compositions (EP 399843; WO 95/17210).

Suitably the metabolisable oil is present in an amount of 0.5% to 20% (final concentration) of the total volume of the immunogenic composition, suitably an amount of 1.0% to 10% of the total volume, suitably in an amount of 2.0% to 6.0% of the total volume.

In a specific embodiment, the metabolisable oil is present in a final amount of about 0.5%, 1%, 3.5% or 5% of the total volume of the immunogenic composition. In another specific embodiment, the metabolisable oil is present in a final amount of 0.5%, 1%, 3.57% or 5% of the total volume of the immunogenic composition. A suitable amount of squalene is about 10.7 mg per vaccine dose, suitably from 10.4 to 11.0 mg per vaccine dose.

Suitably the oil-in-water emulsion systems of the present invention have a small oil droplet size in the sub-micron range. Suitably the droplet sizes will be in the range 120 to 750 nm, suitably sizes from 120 to 600 nm in diameter. Typically the oil-in water emulsion contains oil droplets of which at least 70% by intensity are less than 500 nm in diameter, in particular at least 80% by intensity are less than 300 nm in diameter, suitably at least 90% by intensity are in the range of 120 to 200 nm in diameter.

The oil droplet size, i.e. diameter, according to the present invention is given by intensity. There are several ways of determining the diameter of the oil droplet size by intensity. Intensity is measured by use of a sizing instrument, suitably by dynamic light scattering such as the Malvern Zetasizer 4000 or suitably the Malvern Zetasizer 3000HS. A detailed procedure is given in Example II.2. A first possibility is to determine the z average diameter ZAD by dynamic light scattering (PCS—Photon correlation spectroscopy); this method additionally give the polydispersity index (PDI), and both the ZAD and PDI are calculated with the cumulants algorithm. These values do not require the knowledge of the particle refractive index. A second mean is to calculate the diameter of the oil droplet by determining the whole particle size distribution by another algorithm, either the Contin, or NNLS, or the automatic “Malvern” one (the default algorithm provided for by the sizing instrument). Most of the time, as the particle refractive index of a complex composition is unknown, only the intensity distribution is taken into consideration, and if necessary the intensity mean originating from this distribution.

The oil-in-water emulsion according to the invention may comprise a sterol or a tocopherol, such as alpha tocopherol. Sterols are well known in the art, for example cholesterol is well known and is, for example, disclosed in the Merck Index, 11th Edn., page 341, as a naturally occurring sterol found in animal fat. Other suitable sterols include p-sitosterol, stigmasterol, ergosterol and ergocalciferol. Said sterol is suitably present in an amount of 0.01% to 20% (w/v) of the total volume of the immunogenic composition, suitably at an amount of 0.1% to 5% (w/v). Suitably, when the sterol is cholesterol, it is present in an amount of between 0.02% and 0.2% (w/v) of the total volume of the immunogenic composition, typically at an amount of 0.02% (w/v) in a 0.5 ml vaccine dose volume, or 0.07% (w/v) in 0.5 ml vaccine dose volume or 0.1% (w/v) in 0.7 ml vaccine dose volume.

Suitably alpha-tocopherol or a derivative thereof such as alpha-tocopherol succinate is present. Suitably alpha-tocopherol is present in an amount of between 0.2% and 5.0% (v/v) of the total volume of the immunogenic composition, suitably at an amount of 2.5% (v/v) in a 0.5 ml vaccine dose volume, or 0.5% (v/v) in 0.5 ml vaccine dose volume or 1.7-1.9% (v/v), suitably 1.8% in 0.7 ml vaccine dose volume. By way of clarification, concentrations given in v/v can be converted into concentration in w/v by applying the following conversion factor: a 5% (v/v) alpha-tocopherol concentration is equivalent to a 4.8% (w/v) alpha-tocopherol concentration. A suitable amount of alpha-tocopherol is about 11.9 mg per vaccine dose, suitably from 11.6 to 12.2 mg per vaccine dose.

The oil-in-water emulsion may comprise an emulsifying agent. The emulsifying agent may be present at an amount of 0.01 to 5.0% by weight of the immunogenic composition (w/w), suitably present at an amount of 0.1 to 2.0% by weight (w/w). Suitable concentration are 0.5 to 1.5% by weight (w/w) of the total composition.

The emulsifying agent may suitably be polyoxyethylene sorbitan monooleate (Tween 80). In a specific embodiment, a 0.5 ml vaccine dose volume contains 1% (w/w) Tween 80, and a 0.7 ml vaccine dose volume contains 0.7% (w/w) Tween 80. In another specific embodiment the concentration of Tween 80 is 0.2% (w/w). A suitable amount of polysorbate 80 is about 4.9 mg per vaccine dose, suitably from 4.6 to 5.2 mg per vaccine dose.

Suitably a vaccine dose comprises alpha-tocopherol in an amount of about 11.9 mg per vaccine dose, squalene in an amount of 10.7 mg per vaccine dose, and polysorbate 80 in an amount of about 4.9 mg per vaccine dose.

The oil-in-water emulsion adjuvant may be utilised with other adjuvants or immuno-stimulants and therefore an important embodiment of the invention is an oil in water formulation comprising squalene or another metabolisable oil, a tocopherol, such as alpha tocopherol, and Tween 80. The oil-in-water emulsion may also contain span 85 and/or Lecithin. Typically the oil in water will comprise from 2 to 10% squalene of the total volume of the immunogenic composition, from 2 to 10% alpha tocopherol and from 0.3 to 3% Tween 80, and may be produced according to the procedure described in WO 95/17210. Suitably the ratio of squalene: alpha tocopherol is equal or less than 1 as this provides a more stable emulsion. Span 85 (polyoxyethylene sorbitan trioleate) may also be present, for example at a level of 1%. A suitable example of oil-in-water emulsion adjuvant for use in the invention is given and detailed in EP0399843B, also known as MF59.

Populations to Vaccinate

The target population to vaccinate with the immunogenic compositions of the invention is the entire population, e.g. healthy young adults (e.g. aged 18-60), elderly (typically aged above 60) or infants/children. The target population may in particular be immuno-compromised. Immuno-compromised humans generally are less well able to respond to an antigen, in particular to an influenza antigen, in comparison to healthy adults.

In one aspect according to the invention, the target population is a population which is unprimed against influenza, either being naive (such as vis à vis a pandemic strain), or having failed to respond previously to influenza infection or vaccination. Suitably the target population is elderly persons suitably aged at least 60, or 65 years and over, younger high-risk adults (i.e. between 18 and 60 years of age) such as people working in health institutions, or those young adults with a risk factor such as cardiovascular and pulmonary disease, or diabetes. Another target population is all children 6 months of age and over, who experience a relatively high influenza-related hospitalization rate. In particular, the present invention is suitable for a paediatric use in children between 6 months and 3 years of age, or between 3 years and 8 years of age, such as between 4 years and 8 years of age, or between 9 years and 17 years of age. Accordingly, in one embodiment of the invention, there is provided an immunogenic composition comprising an antigen or an antigenic preparation from a first influenza virus strain and an oil-in-water emulsion adjuvant for use in inducing an immune response against at least one second influenza virus strain, which is of a type or a subtype different from the first influenza virus strain, in subjects between 6 months and 3 years of age, or between 4 years and 8 years of age, or between 9 years and 17 years of age. In a specific embodiment, there is provided an immunogenic composition comprising an antigen or an antigenic preparation from a first influenza virus strain and an oil-in-water emulsion adjuvant for use in inducing an immune response against at least one second influenza virus strain, which is of a type or a subtype different from the first influenza virus strain, in subjects being ≧3 years of age.

Revaccination and Composition Used for Revaccination

An aspect of the present invention provides an influenza immunogenic composition for revaccination of humans previously vaccinated with an immunogenic influenza composition formulated with an oil-in-water emulsion adjuvant, as well as a method of prevention and/or treatment against influenza disease, wherein a first immunogenic composition comprising an antigen or an antigenic preparation from at least one influenza virus strain together with an oil-in-water emulsion adjuvant is first administered and a second immunogenic composition comprising an antigen or an antigenic preparation from at least one influenza virus strain is administered. In the sense of the present invention, the terms “administration of a second immunogenic composition” and “revaccination” are to be considered as synonyms, and will be used in an interchangeable way. Typically revaccination is made at least 6 months after the first vaccination(s), suitably 8 to 14 months after, suitably at around 10 to 12 months after.

The immunogenic composition for revaccination may contain any type of antigen preparation, either inactivated, recombinant or live attenuated. It may contain the same type of antigen preparation i.e. split influenza virus or split influenza virus antigenic preparation thereof, a whole virion, a purified HA and NA (sub-unit) vaccine or a virosome, as the immunogenic composition used for the first vaccination. Alternatively the second composition may contain another type of influenza antigen, i.e. split influenza virus or split influenza virus antigenic preparation thereof, a whole virion, a purified HA and NA (sub-unit) vaccine or a virosome, than that used for the first vaccination. Suitably a split virus or a whole virion vaccine is used.

The second immunogenic composition may be adjuvanted or un-adjuvanted. In one embodiment the second immunogenic composition is not adjuvanted and is a classical influenza vaccine containing three inactivated split virion antigens prepared from the WHO recommended strains of the appropriate influenza season, such as Fluarix™/α-Rix®/Influsplit® given intramuscularly.

In another embodiment, the second immunogenic composition is adjuvanted, e.g. adjuvanted with any of the adjuvant described above, e.g. oil-in-water adjuvants. Suitably, the second immunogenic composition comprises an oil-in-water emulsion adjuvant, in particular an oil-in-water emulsion adjuvant comprising a metabolisable oil, optionally a sterol or a tocopherol, such as alpha tocopherol, and an emulsifying agent. Specifically, said oil-in-water emulsion adjuvant comprises at least one metabolisable oil in an amount of 0.5% to 20% of the total volume, and has oil droplets of which at least 70% by intensity have diameters of less than 1 μm. Alternatively the second immunogenic composition comprises an alum adjuvant, either aluminium hydroxide or aluminium phosphate or a mixture of both.

In one embodiment, the first vaccination is made with a pandemic influenza composition as previously described, suitably a split influenza composition, and the re-vaccination is made as follows.

In an embodiment according to the invention, the second immunogenic composition is a monovalent influenza composition comprising an influenza virus strain which is associated with a pandemic or has the potential to be associated with a pandemic. Suitable strains are, but not limited to: H5N1, H9N2, H7N7, H2N2, H7N1 and H1N1. Said strain may be the same as that, or one of those, present in the composition used for the first vaccination. In an alternative embodiment said strain may be a variant strain, i.e. a drifted strain or a shifted strain, of the strain present in the composition used for the first vaccination.

In another specific embodiment, the second immunogenic composition for re-vaccination is a multivalent influenza vaccine. In particular, when the boosting composition is a multivalent vaccine such as a bivalent, trivalent or quadrivalent vaccine, at least one strain is associated with a pandemic or has the potential to be associated with a pandemic. In a specific embodiment, two or more strains in the second immunogenic composition are pandemic strains. In another specific embodiment, the at least one pandemic strain in the second immunogenic composition is of the same type as that, or one of those, present in the composition used for the first vaccination. In an alternative embodiment the at least one strain may be a variant strain, i.e. a drifted strain or a shifted strain, of the at least one pandemic strain present in the composition used for the first vaccination.

Suitably, the second immunogenic composition, where used, is given at the next influenza season, e.g. approximately one year after the first immunogenic composition. The second immunogenic composition may also be given every subsequent year (third, fourth, fifth vaccination and so forth). The second immunogenic composition may be the same as the composition used for the first vaccination. Suitably, the second immunogenic composition contains an influenza virus or antigenic preparation thereof which is a variant strain of the influenza virus used for the first vaccination. In particular, the influenza viral strains or antigenic preparation are selected according to the reference material distributed by the World Health Organisation such that they are adapted to the influenza virus strain which is circulating on the year of the revaccination. Suitably the first vaccination is made at the declaration of a pandemic and re-vaccination is made later. Suitably, the revaccination is made with a vaccine comprising an influenza virus strain (e.g. H5N1 Vietnam) which is of the same subtype as that used for the first vaccination (e.g. H5N1 Vietnam). In a specific embodiment, the revaccination is made with a drifted strain of the same sub-type, e.g. H5N1 Indonesia. In another embodiment, said influenza virus strain used for the revaccination is a shifted strain, i.e. is different from that used for the first vaccination, e.g. it has a different HA or NA subtype, such as H5N2 (same HA subtype as H5N1 but different NA subtype) or H7N1 (different HA subtype from H5N1 but same NA subtype).

Suitably revaccination induces any, suitably two or all, of the following: (i) an improved CD4 response against the influenza virus or antigenic preparation thereof, or (ii) an improved B cell memory response or (iii) an improved humoral response, compared to the equivalent response induced after a first vaccination with the un-adjuvanted influenza virus or antigenic preparation thereof. Suitably the immunological responses induced after revaccination with the adjuvanted influenza virus or antigenic preparation thereof as herein defined, are higher than the corresponding response induced after the revaccination with the un-adjuvanted composition. Suitably the immunological responses induced after revaccination with an un-adjuvanted, suitably split, influenza virus are higher in the population first vaccinated with the adjuvanted, suitably split, influenza composition than the corresponding response in the population first vaccinated with the un-adjuvanted, suitably split, influenza composition.

The adjuvanted composition of the invention will be capable of inducing a better cross-responsiveness against drifted strain (the influenza virus strain from the next influenza season) compared to the protection conferred by the control vaccine. Said cross-responsiveness has shown a higher persistence compared to that obtained with the un-adjuvanted formulation. The effect of the adjuvant in enhancing the cross-responsiveness against drifted strain is of importance in a pandemic situation.

In a further embodiment the invention relates to a vaccination regime in which the first vaccination is made with an influenza composition, suitably a split influenza composition, containing an influenza virus strain that could potentially cause a pandemic and the revaccination is made with a composition, either monovalent or multivalent, comprising at least one circulating strain, either a pandemic strain or a classical strain, possibly strains of a subtype or type different from the strain(s) used for the first vaccination.

Vaccination Means

The composition of the invention may be administered by any suitable delivery route, such as intradermal, mucosal e.g. intranasal, oral, intramuscular or subcutaneous. Other delivery routes are well known in the art.

The intramuscular delivery route is particularly suitable for the adjuvanted influenza composition. The composition according to the invention may be presented in a monodose container, or alternatively, a multidose container, particularly suitable for a pandemic vaccine. In this instance an antimicrobial preservative such a thiomersal may be present to prevent contamination during use. A thiomersal concentration of 5 μg/0.5 ml dose (i.e. 10 μg/ml) or 10 μg/0.5 ml dose (i.e. 20 μg/ml) is suitably present. A suitable IM delivery device could be used such as a needle-free liquid jet injection device, for example the Biojector 2000 (Bioject, Portland, Oreg.). Alternatively a pen-injector device, such as is used for at-home delivery of epinephrine, could be used to allow self administration of vaccine. The use of such delivery devices may be particularly amenable to large scale immunization campaigns such as would be required during a pandemic.

Intradermal delivery is another suitable route. Any suitable device may be used for intradermal delivery, for example short needle devices. Such devices are well known in the art. Intradermal vaccines may also be administered by devices which limit the effective penetration length of a needle into the skin, such as those described in WO99/34850 and EP1092444, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Also suitable, are ballistic powder/particle delivery devices which use compressed gas to accelerate vaccine in powder form through the outer layers of the skin to the dermis. Additionally, conventional syringes may be used in the classical mantoux method of intradermal administration.

Another suitable administration route is the subcutaneous route. Any suitable device may be used for subcutaneous delivery, for example classical needle. Suitably, a needle-free jet injector service is used. Such devices are well known in the art. Suitably said device is pre-filled with the liquid vaccine formulation.

Alternatively the vaccine is administered intranasally. Typically, the vaccine is administered locally to the nasopharyngeal area, suitably without being inhaled into the lungs. It is desirable to use an intranasal delivery device which delivers the vaccine formulation to the nasopharyngeal area, without or substantially without it entering the lungs.

Suitable devices for intranasal administration of the vaccines according to the invention are spray devices. Suitable commercially available nasal spray devices include Accuspray™ (Becton Dickinson). Nebulisers produce a very fine spray which can be easily inhaled into the lungs and therefore does not efficiently reach the nasal mucosa. Nebulisers are therefore not preferred.

Suitable spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are described for example in WO 91/13281 and EP 311 863 B and EP 516 636, incorporated herein by reference. Such devices are commercially available from Pfeiffer GmbH and are also described in Bommer, R. Pharmaceutical Technology Europe, September 1999.

Suitable intranasal devices produce droplets (measured using water as the liquid) in the range 1 to 200 μm, suitably 10 to 120 μm. Below 10 μm there is a risk of inhalation, therefore it is desirable to have no more than about 5% of droplets below 10 μm. Droplets above 120 82 m do not spread as well as smaller droplets, so it is desirable to have no more than about 5% of droplets exceeding 120 μm.

Alternatively, the epidermal or transdermal vaccination route is also contemplated in the present invention.

In one aspect of the present invention, the adjuvanted immunogenic composition for the first administration may be given intramuscularly, and the boosting composition, either adjuvanted or not, may be administered through a different route, for example intradermal, subcutaneous or intranasal. In a specific embodiment, the composition for the first administration contains a HA amount of less than 15 μg for the pandemic influenza virus strain, and the boosting composition may contain a standard amount of 15 μg or, suitably a low amount of HA, i.e. below 15 μg, which, depending on the administration route, may be given in a smaller volume.

Vaccination Regimes, Dosing and Efficacy Criteria

Suitably, the immunogenic compositions for use according to the present invention are a standard 0.5 ml injectable dose in most cases, and contain 15 μg, or less, of haemagglutinin antigen component from an influenza virus strain, as measured by single radial immunodiffusion (SRD) (J. M. Wood et al.: J. Biol. Stand. 5 (1977) 237-247; J. M. Wood et al., J. Biol. Stand. 9 (1981) 317-330). Suitably the vaccine dose volume will be between 0.5 ml and 1 ml, in particular a standard 0.5 ml, or 0.7 ml vaccine dose volume. Slight adaptation of the dose volume will be made routinely depending on the HA concentration in the original bulk sample and depending also on the delivery route with smaller doses being given by the intranasal or intradermal route.

Suitably said immunogenic compositions for use according to the invention contain a low amount of HA antigen—e.g any of 1, 2, 3, 4, 5, 6, 7, 8, 9, 10, 11, 12, 13, 14 μg of HA per influenza virus strain or which does not exceed 15 μg of HA per strain. Said low amount of HA amount may be as low as practically feasible provided that it allows to formulate a vaccine which meets the international e.g. EU or FDA criteria for efficacy, as detailed below (see Table 1 and the specific parameters as set forth). A suitable low amount of HA is between 1 to 7.5 μg of HA per influenza virus strain, suitably between 3.5 to 5 μg such as 3.75 or 3.8 μg of HA per influenza virus strain, typically about 5 μg of HA per influenza virus strain. Another suitable amount of HA is between 0.1 and 5 μg of HA per influenza virus strain, suitably between 1.0 and 2 μg of HA per influenza virus strain such as 1.9 μg of HA per influenza virus strain.

Advantageously, a vaccine dose according to the invention, in particular a low HA amount vaccine, may be provided in a smaller volume than the conventional injected split flu vaccines, which are generally around 0.5, 0.7 or 1 ml per dose. The low volume doses according to the invention are suitably below 500 μl, typically below 300 μl and suitably not more than about 200 μl or less per dose.

Thus, a suitable low volume vaccine dose according to one aspect of the invention is a dose with a low antigen dose in a low volume, e.g. about 15 μg or about 7.5 μg HA or about 3.0 μg HA (per strain) in a volume of about 200 μl.

The influenza medicament of the invention suitably meets certain international criteria for vaccines. Standards are applied internationally to measure the efficacy of influenza vaccines. Serological variables are assessed according to criteria of the European Agency for the Evaluation of Medicinal Products for human use (CHMP/BWP/214/96, Committee for Proprietary Medicinal Products (CPMP). Note for harmonization of requirements for influenza vaccines, 1997. CHMP/BWP/214/96 circular No 96-0666:1-22) for clinical trials related to annual licensing procedures of influenza vaccines (Table 1). The requirements are different for adult populations (18-60 years) and elderly populations (>60 years) (Table 1). For interpandemic influenza vaccines, at least one of the assessments (seroconversion factor, seroconversion rate, seroprotection rate) should meet the European requirements, for all strains of influenza included in the vaccine. The proportion of titres equal or greater than 1:40 is regarded most relevant because these titres are expected to be the best correlate of protection [Beyer W et al. 1998. Clin Drug Invest.; 15:1-12].

As specified in the “Guideline on dossier structure and content for pandemic influenza vaccine marketing authorisation application. (CHMP/VEG/4717/03, Apr. 5, 2004), in the absence of specific criteria for influenza vaccines derived from non circulating strains, it is anticipated that a pandemic candidate vaccine should (at least) be able to elicit sufficient immunological responses to meet suitably all three of the current standards set for existing vaccines in unprimed adults or elderly subjects, after two doses of vaccine.

The compositions for use according to the present invention suitably meet at least one such criteria for the influenza virus strain included in the composition (one criteria is enough to obtain approval), suitably at least two, or typically at least all three criteria for protection as set forth in Table 1.

TABLE 1
(CHMP criteria)

| | 18-60 years | >60 years |
|---|---|---|
| Seroconversion rate* | >40% | >30% |
| Conversion factor** | >2.5 | >2.0 |
| Protection rate*** | >70% | >60% |

*Seroconversion rate is defined as the proportion of subjects in each group having a protective post-vaccination titre ≧1:40. The seroconversion rate simply put is the % of subjects who have an HI titre before vaccination of <1:10 and ≧1:40 after vaccination. However, if the initial titre is ≧1:10 then there needs to be at least a fourfold increase in the amount of antibody after vaccination.
**Conversion factor is defined as the fold increase in serum HI geometric mean titres (GMTs) after vaccination, for each vaccine strain.
***Protection rate is defined as the proportion of subjects who were either seronegative prior to vaccination and have a (protective) post-vaccination HI titre of ≧1:40 or who were seropositive prior to vaccination and have a significant 4-fold increase in titre post-vaccination; it is normally accepted as indicating protection.

A 70% seroprotection rate is defined by the European health regulatory authority (CHMP—Committee for Medicinal Products for Human Use) is one of three criteria normally required to be met for an annual seasonal influenza vaccine and which CHMP is also expecting a pandemic candidate vaccine to meet. However, mathematical modelling has indicated that a vaccine that is only 30% efficient against certain drifted strains may also be of benefit in helping to reduce the magnitude of a pandemic (Ferguson et al, Nature 2006).

FDA has published a draft guidance (available from the Office of Communication, Training and Manufacturers Assistance (HFM-40), 1401 Rockville Pike, Suite 200N, Rockville, Md. 20852-1448, or by calling 1-800-835-4709 or 301-827-1800, or from the Internet at http://www.fda.gov/cber/guidelines.htm) on Clinical Data Needed to Support the Licensure of Pandemic Influenza Vaccines, and the proposed criteria are also based on the CHMP criteria. Appropriate endpoints similarly include: 1) the percent of subjects achieving an HI antibody titer ≧1:40, and 2) rates of seroconversion, defined as a four-fold rise in HI antibody titer post-vaccination. The geometric mean titer (GMT) should be included in the results. These data and the 95% confidence intervals (CI) of the point estimates of these evaluations should be provided.

Accordingly, in one aspect of the invention, it is provided for a composition, method or use as claimed herein wherein said immune response or protection induced by the administration of the contemplated immunogenic compositions meets all three EU regulatory criteria for influenza vaccine efficacy. Suitably at least one, suitably two, or three of following criteria are met for the influenza virus strains of the composition:
a seroconversion rate of >50%, of >60%, of >70%, suitably of >80% or >90% in the adult population (aged 18-60), and/or suitably also in the elderly population (aged >60 years);
a protection rate of >75%, of >80%, of >85%, suitably of >90% in the adult population (aged 18-60), and/or suitably also in the elderly population (aged >60 years);
a conversion factor of >4.0, of >5.0, of >6.0, of >7.0, of >8.0, of >9.0 or of 10 or above 10 in the adult population (aged 18-60), and/or suitably also in the elderly population (aged >60 years).

In a specific embodiment the composition for use according to the invention will meet both a seroconversion rate of >60%, or >70%, or suitably >80% and a protection rate of >75%, suitably of >80% in the adult population. In another specific embodiment the composition according to the invention will meet both a conversion factor of >5.0, or >7.0 or suitably >10.0 and a seroconversion rate of >60%, or >70%, or suitably >80% in the adult population. In another specific embodiment, the composition according to the invention will meet both a conversion factor of >5.0, or >7.0 or suitably >10.0, and a protection rate of >75%, suitably >80% in the adult population. In still another specific embodiment the composition according to the invention will meet both a conversion factor of 10.0 or above, a seroconversion rate of 80% or above, and a protection rate of 80% or above.

In another embodiment, the compositions for use according to the invention will meet a seroprotection rate of at least 30% against drifted strains, suitably of at least 40%, or >50% or >60% against drifted strains. Suitably the seroprotection rate will be >70%, or suitably >80% against drifted strains.

Suitably any or all of such criteria are also met for other populations, such as in children and in any immuno-compromised population.

Suitably the above response(s) is(are) obtained after one dose, or typically after two doses. It is a particular advantage of compositions for use according to the invention that the immune response is obtained after only one dose of adjuvanted vaccine. Accordingly, there is provided in one aspect of the invention the use of a non-live influenza virus antigen preparation, possibly from a pandemic strain, in particular a split influenza virus preparation, for a one-dose vaccination against influenza, wherein the one-dose vaccination generates an immune response which meets at least one, suitably two or three, international regulatory requirements for influenza vaccines. In a particular embodiment said immune response is a cross-reactive antibody response or a cross-reactive CD4 T cell response or both. In a specific embodiment, the human patient is immunologically naive (i.e. does not have pre-existing immunity) to the vaccinating strain. Specifically the composition for use according to the invention contains a low HA antigen amount.

In respect of the composition for re-vaccination, when it is a multivalent composition, at least two or all three of the criteria will need to be met for all strains, particularly for a new vaccine. Under some circumstances two criteria may be sufficient. For example, it may be acceptable for two of the three criteria to be met by all strains while the third criterion is met by some but not all strains (e.g. two out of three strains).

The teaching of all references in the present application, including patent applications and granted patents, are herein fully incorporated by reference. Any patent application to which this application claims priority is incorporated by reference herein in its entirety in the manner described herein for publications and references.

For the avoidance of doubt the terms ‘comprising’, ‘comprise’ and ‘comprises’ herein is intended by the inventors to be optionally substitutable with the terms ‘consisting of’, ‘consist of’, and ‘consists of’, respectively, in every instance.

The invention will be further described by reference to the following, non-limiting, examples:

### EXAMPLE 1

### Assays for Assessing the Immune Response in Humans

### 1.1 Hemagglutination Inhibition Assay

The immune response was determined by measuring HI antibodies using the method described by the WHO Collaborating Centre for influenza, Centres for Disease Control, Atlanta, USA (1991). Antibody titre measurements were conducted on thawed frozen serum samples with a standardised and comprehensively validated micromethod using 4 hemagglutination-inhibiting units (4 HIU) of the appropriate antigens and a erythrocyte suspension. Non-specific serum inhibitors were removed by receptor-destroying enzyme followed by heat inactivation. The sera obtained were evaluated for HI antibody levels. Starting with an initial dilution of 1:10, a dilution series (by a factor of 2) was prepared up to an end dilution of 1:20480. The titration end-point was taken as the highest dilution step that showed complete inhibition (100%) of hemagglutination. All assays were performed in duplicate.

### 1.2. Neutralising Antibody Assay

Neutralising antibody measurements were conducted on thawed frozen serum samples. Virus neutralisation by antibodies contained in the serum is determined in a microneutralization assay. The sera are used after heat inactivation 30 min at 56° C. Each serum is tested in triplicate. A standardised amount of virus is mixed with serial dilutions of serum and incubated to allow binding of the antibodies to the virus. A cell suspension, containing a defined amount of Madin-Darby Canine Kidney (MDCK) cells is then added to the mixture of virus and antiserum and incubated at 37° C. After the incubation period, virus replication is visualised by hemagglutination of chicken red blood cells. The 50% neutralisation titre of a serum is calculated by the method of Reed and Muench (Am. J; Hyg. 1938, 27: 493-497).

### 1.3 Statistical Methods

1.3.1 For the humoral immune response in terms of HI antibodies against H1N1 (in all subjects in the TIV Group), the following parameters will be calculated with 95% CIs:

Observed Variable:
H1N1 HI antibody titres on Day 0 and Day 28.

Derived Variables:
GMTs and seropositivity rates on Day 0 and Day 28;
Seroprotection rates (SPRs) on Day 0 and Day 28.
Seroconversion rate (SCR) on Day 28
Mean Geometric Increase (MGI) on Day 28
SPR is defined as the percentage of vaccinees with a serum HI titre >=1:40 that usually is accepted as indicating protection

SCR for HI antibody response is defined as the percentage of vaccinees that have either a pre-vaccination (Day 0) titre <1:10 and a post-vaccination titre >=1:40 or a pre-vaccination titre >=1:10 and at least a 4-fold increase in post-vaccination titre.

MGI is defined as the geometric mean of the within-subject ratios of the post-vaccination reciprocal HI titer to the pre-vaccination (Day 0) reciprocal HI titer.

GMT is for geometric mean titer

### 1.3.2 Solicited Local and General Adverse Events:

Occurrence, intensity and duration of each solicited local and general AE (any and grade 3) within 7 days (Day 0-Day 6) after each vaccination.

Unsolicited Adverse Events:
Occurrence, intensity and relationship to vaccination of unsolicited AEs within 28 days (Day 0-Day 27) after each vaccination, according to the Medical Dictionary for Regulatory Activities (MedDRA) classification. MAEs/AESIs/pIMDs/SAEs: and AEs of special interest
Occurrence of MAEs, AESIs/pIMDs, SAEs and AEs of special interest and relationship to additional vaccination during the entire study period.
   For the humoral immune response in terms of HI antibodies against all TIV strains in all subjects and per age strata, the following parameters will be calculated with 95% CIs:

Observed Variable:
HI antibodies on Day 0, Day 28*, and Month 6**. *TIV Group only**only H1N1 in the Control group

Derived Variables:
GMTs and seropositivity rates on Day 0, Day 28*, and Month 6**;
SCRs on Day 28*, and Month 6**;
SPRs on Day 0, Day 28*, and Month 6**;
MGIs on Day 28*, and Month 6**.
   For the humoral immune response in terms of neutralising antibodies against all TIV strains, the following parameters will be calculated with 95% CI (in a subset of subjects):

Observed Variable:
Serum neutralising antibody titres on Day 0, Day 28*, and Month 6**.

Derived Variables:
GMTs of serum neutralising antibody titres and seropositivity rates;
SCRs.

### EXAMPLE 2

### Immunogenicity Studies

### 2.1 Statistical Methods

Study 1: A Phase IV, open label, randomized, multicountry study to evaluate immunogenicity and safety of GSK Biologicals' seasonal (2010-2011) influenza vaccine Fluarix™ children (6M to <9Y) previously vaccinated with GSK Biologicals' H1N1 vaccine (Pandemrix™). Pandemrix™ contains oil-in-water emulsion adjuvant AS03, which is composed of squalene, DL-alpha-tocopherol and polysorbate 80.

Study 2: A Phase IV, open label, randomized, monocentric study to evaluate immunogenicity and safety of GSK Biologicals' seasonal (2010-2011) influenza vaccine Fluarix™ adolescents (10-17Y) previously vaccinated with GSK Biologicals' H1N1 vaccine (Pandemrix™).

The Vaccine strain homologous immune responses as detected by hemagglutination inhibition and microneutralization tests are humoral immune responses (i.e. anti-hemagglutinin, neutralising) measured at Day 0, Day 28 and at Month 6.

### 2.2 Study Design

Study 1: 154 subjects 6 months to 9 years of age when they were vaccinated with two 0.25 mL doses of H1N1 adjuvanted vaccine (Pandemrix™) were enrolled.

Enrollment was Stratified as Follows:
6-11 months old at the time of first vaccination with Pandemrix™.
12-35 months at the time of first vaccination with Pandemrix™.
3-9 years old at the time of first vaccination with Pandemrix™.

Study 2: 77 between 10-17 years of age when they were vaccinated with one dose of H1N1 adjuvanted vaccine (Pandemrix™) were enrolled.

The Fluarix™ vaccine was administered in the deltoid region of the non-dominant arm on Day 0 and Day 28(if applicable).
Dosage: All subjects: 0.5 mL.
Number of doses: Primed subjects are subjects previously vaccinated with seasonal flu vaccine, based on vaccination history
   Children >=9 years and primed children <9 years: one dose.
   Unprimed children 6 months to <9 years: two doses with at least a 4-week interval.

As a non-influenza vaccine control, a first dose of hepatitis A vaccine (Havrix™) was administered, with the second dose to complete the vaccination course given outside the study setting at the Month 6 visit.

Treatment Groups:

TIV Group: Subjects previously vaccinated with adjuvanted H1N1 vaccine received one dose of TIV vaccine Fluarix™ (in accordance with the SmPC).

Control Group: Subjects previously vaccinated with adjuvanted H1N1 vaccine received one first dose of Havrix (dose 2 given as recommended per SmPC, outside the study setting, at Month 6).
Subjects aged <15 years received Havrix Junior (720 ELISA Units/0.5 ml dose)
Subjects aged >15 years will receive Havrix (1440 ELISA Units/1 ml dose)

Blood Sampling Schedule:

TIV Group: Blood samples on Day 0, Day 28, and Month 6.

Control Group: Blood samples on Day 0 and Month 6.

### 2.3 Study Objectives

Study 1: To evaluate HI immune response against the H1N1 strain 28 days following vaccination with the first dose of trivalent inactivated influenza virus (TIV) vaccine

(Fluarix™) in subjects previously vaccinated with 2 doses of H1N1 adjuvanted vaccine (Pandemrix™).

Study 2: To evaluate HI immune response against the H1N1 strain 28 days following vaccination with TIV vaccine (Fluarix™) in subjects previously vaccinated with 1 dose of H1N1 adjuvanted vaccine (Pandemrix™) in the TIV Group.
To evaluate safety and reactogenicity after each flu vaccination.
To assess the vaccine immune response in terms of HI (in all subjects) and neutralising antibodies (in a subset of subjects) against the 3 TIV strains, 28 days after the first dose of TIV vaccine overall and per age strata, in the TIV group.
To assess the immune status at the pre-vaccination time point in terms of HI (in all subjects) and neutralising (in a subset of subjects) antibodies against the 3 TIV strains per age strata in both study groups.
To assess the persistence of antibodies against the 3 TIV strains 6 months after the first TIV vaccine dose in terms of HI (in all subjects) and neutralising (in a subset of subjects) antibodies in the TIV group.
To assess the persistence of the immune response at the month 6 time point in terms of HI (in all subjects) and neutralising (in a subset of subjects) antibodies against the H1N1 strain in the control group.

### 2.4 Study Population Results

Study 1: Number of Subjects:

Planned: 360 subjects, 180 in each group

Enrolled: 162 subjects, 81 in the TIV Group and 80 in the Control Group, and one subject who was not assigned to any group (due to withdrawal before randomisation).

Completed up to Month 6: 144 subjects, 68 in the TIV Group and 76 in the Control Group.

Safety up to Month 6: 154 subjects were included in the Total Vaccinated cohort (TVC) (77 in the TIV Group and 77 in the Control Group).

Immunogenicity up to Month 6: 126 subjects were included in the according-to-protocol (ATP) cohort for persistence at Month 6 (56 in the TIV Group and 70 in the Control Group).

Study 2: Number of Subjects:

Planned: 120 subjects, 60 in each group.

Enrolled: 77 subjects, 38 in the TIV Group and 39 in the Control Group.

Completed at Month 6: 75 subjects, 36 in the TIV Group and 39 in the Control Group.

Safety: 77 subjects were included in the Total vaccinated cohort (38 in the TIV Group and 39 in the Control Group)

Immunogenicity: 72 subjects were included in the According-to-protocol (ATP) cohort for analysis of antibody persistence (35 in the TIV Group and 37 in the Control Group).

### 2.5 Safety Conclusions

The administration of influenza vaccine Fluarix™ in children and adolescents previously vaccinated with GSK Biologicals' H1N1 vaccine Pandemrix™ elicited a clinically acceptable profile of adverse events with no safety concerns

### 2.6 Immunogenicity Results

The administration of Fluarix™ vaccine to children and adolescents who had previously been vaccinated with Pandemrix™ resulted in persistence of HI response at six months for each strain contained in the Fluarix™ vaccine (A/California[H1N1]v-like, B/Brisbane and A/Victoria)

TABLE 2
Clinical Immunogenicity Results

| | | GMT (SPR) | GMT (SPR) |
|---|---|---|---|
| | | (6 mo-9 yrs; | (10-17 yrs; |
| Strain | Timing | N = 56) | N = 35) |
| FluA/CAL/7/ | Day 0 | 120.7 | 150.1 |
| 09 (H1N1) HA Ab | Day 28 | 1079.3 | 646.8 |
| | Month 6 | 509 (100%) | 346.4 (100%) |
| FluB/Bri/60/08 | Day 0 | 17.4 | 22.2 |
| (Victoria) HA Ab | Day 28 | 160.9 | 320.1 |
| | Month 6 | 154.1 (92.9%) | 242.4 (94.3%) |
| FluA/Victoria/210/09 | Day 0 | 20.8 | 20.0 |
| (H3N2) HA Ab | Day 28 | 396.3 | 279.2 |
| | Month 6 | 186.8 (100%) | 160.1 (97.1%) |

GMT is for geometric mean titer

### EXAMPLE 3

### Confirmation of H1N1 Priming in a Pre-Clinical Mouse Model

### 3.1 Study Design and Methods

In order to confirm the priming effects observed in the human studies described in Example 2, a preclinical mouse model was employed, according to the study design shown in Table 3. Six- to eight-week old female BALB/c mice (Charles River Canada) were immunized intramuscularly in a hind limb (50 μL of vaccine or PBS per injection) on Days 0 and 28 or 91 without anaesthesia. Animals were first immunized with 0.375 μg (1/10 full human dose (FHD)) or 0.075 μg HA (1/50 FHD) of Pandemrix™ (Groups 1 to 8) and then with 1.5 μg (1/10 FHD) or 0.3 μg HA (1/50 FHD) of Fluarix™ (Groups 1 to 8). Control animals were immunized with 1.5 μg HA (1/10 FHD) of Fluarix™ or PBS twice (Group 9 and 10 respectively). Mice were bled 28 days post-prime and 21 and 49 days post-boost to measure serum HI antibody responses using the Hemagglutination Inhibition (HI) Assay described in Example 1.

TABLE 3
120 mice were randomly assigned to
one of the following study groups:

| | | Treatment-Prime | | | |
|---|---|---|---|---|---|
| | | (Pandemrix ™ except | | | |
| | | group 9: Fluarix ™, | | | |
| | | and group 10: PBS) | | Treatment-Boost | |
| | | Vaccine | | (Fluarix ™) | Prime and |
| | | dose | Adjuvant | Vaccine dose | Boost |
| Group | N | (μg HA) | dose | (μg HA) | schedule |
| 1 | 12 | 0.375 | AS03 | 1.5 | Day 0 and |
| 2 | | 0.375 | | 0.3 | Day 28 |
| 3 | | 0.075 | | 1.5 | |
| 4 | | 0.075 | | 0.3 | |
| 5 | | 0.375 | | 1.5 | Day 0 and |
| 6 | | 0.375 | | 0.3 | Day 91 |
| 7 | | 0.075 | | 1.5 | |
| 8 | | 0.075 | | 0.3 | |
| 9 | | 1.5 | None | 1.5 | Day 0 and |
| | | Fluarix ™ | | Fluarix ™ | Day 28 |
| 10 | | PBS | | PBS | |

N: Number of mice per group
0.375 μg HA for Pandemrix ™ represents 1/10 full human dose (FHD)
0.075 μg HA for Pandemrix ™ represents 1/50 FHD
1.5 μg HA/strain for Fluarix ™ represents 1/10 FHD
0.3 μg HA/strain for Fluarix ™ represents 1/50 FHD

### 3.2 Results

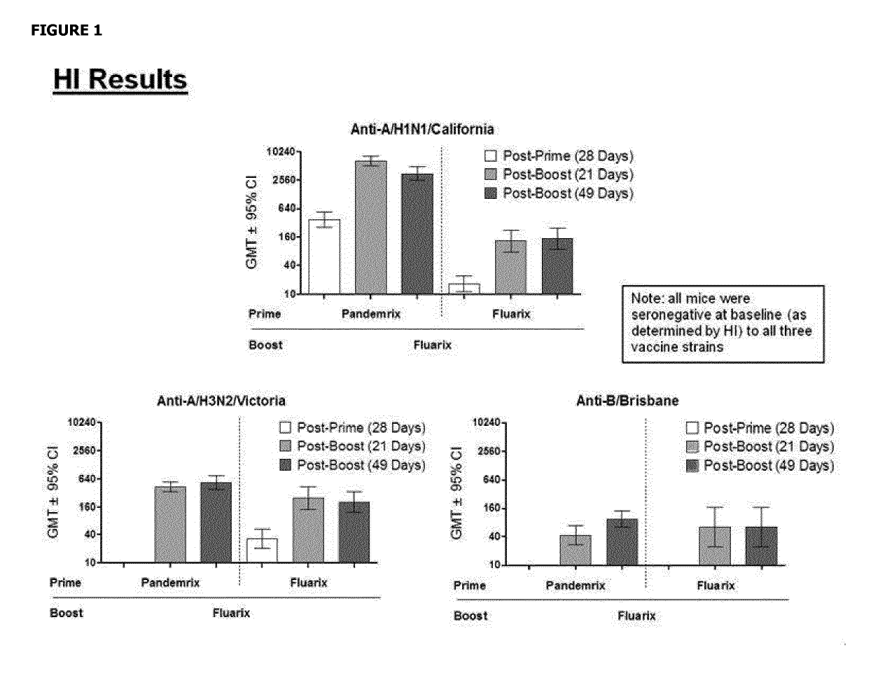

The clinical observations described in Example 2 were reproducible in a mouse model of immunogenicity. Specifically, priming with Pandemrix™ followed by Fluarix™ boost gave higher HI titers against A/H3N2/Victoria and B/Brisbane (and A/H1N1/California) compared to one administration of Fluarix™ (FIG. 1). The results were independent of the prime-boost schedule (28 or 91 days apart). Titers persisted at least to Day 49 post-boost. Priming with Pandemrix™ followed by Fluarix™ boost gave higher HI titers against A/H1N1/California compared to Fluarix™ prime-boost. Priming with Pandemrix™ followed by Fluarix™ boost gave comparable HI titers against A/H3N2/Victoria and B/Brisbane compared to Fluarix™ prime-boost.

1. A method of inducing an immune response against at least one second influenza virus strain wherein said second influenza virus strain is from a different type or from a different subtype than a first influenza virus strain, the method comprising: administering to a subject an immunogenic composition comprising an antigen or an antigenic preparation from a first influenza virus strain and an oil-in-water emulsion adjuvant.

2. The method of claim 1, wherein said first influenza virus strain is of a A-type.

3. The method of claim 1, wherein said first influenza virus strain is of a B-type.

4. The method of claim 1, wherein said composition comprises an antigen or an antigenic preparation from multiple influenza virus strains.

5. The method of claim 1, wherein said second influenza virus strain is of a A-type or of a B-type.

6. The method of claim 1, wherein said method induces an immune response against multiple influenza virus strains.

7. The method of claim 1, wherein said method induces an immune response against one, two, three or all, of: an A/H1N1 strain, an A/H3N2 strain, a B strain of the Yagamata lineage and a B strain of the Victoria lineage.

8. The method of claim 1, wherein the induced immune response persists for at least 6 months.

9. The method of claim 1, wherein said antigen is haemagglutinin.

10.-11. (canceled)

12. The method of claim 1, wherein said composition is monovalent.

13. The method of claim 1, wherein said composition is multivalent.

14. The method of claim 1, wherein said oil-in-water emulsion adjuvant comprises a metabolisable oil, and a emulsifying agent.

15. The method of claim 14, wherein said oil-in-water emulsion adjuvant further comprises alpha-tocopherol.

16.-17. (canceled)

18. The method of claim 1, wherein said immunogenic composition is administered parenterally.

19.-20. (canceled)

21. A method of immunization according to a one dose scheme in a paediatric subject which has previously been vaccinated with a first immunogenic composition comprising an antigen or an antigenic preparation from at least one influenza virus strain and oil-in-water emulsion adjuvant, the method comprising: administering to the subject a second immunogenic composition comprising an antigen or an antigenic preparation from at least one influenza virus strain.

22. The method of claim 21, wherein the at least one influenza virus strain of the second immunogenic composition is of a type or a subtype different from the at least one influenza virus strain of the first immunogenic composition.

23. The method of claim 21, wherein said second composition is unadjuvanted.

24. The method of claim 21, wherein said second composition is a trivalent composition comprising two A-type influenza virus strains of different subtypes and one B-type influenza virus strain.

25. The method of claim 21, wherein the paediatric subjects are between 6 months and 3 years of age, or between 4 years and 8 years of age.

26. (canceled)

27. The method of claim 21, wherein said paediatric subject has been vaccinated with the first immunogenic composition one year before being administered the second immunogenic composition.

28.-29. (canceled)

30. A method of prevention or treatment against influenza disease, the method comprising the administration of a first immunogenic composition comprising an antigen or an antigenic preparation from at least one influenza virus strain together with an oil-in-water emulsion adjuvant, followed by the administration of a second immunogenic composition comprising an antigen or an antigenic preparation from at least one influenza virus strain, wherein the administration of the first immunogenic composition induces an immune response against an influenza virus strain included in the second immunogenic composition, but not present in the first immunogenic composition.

31. The method of claim 30, wherein the at least, one influenza virus strains of the first immunogenic composition and of the second immunogenic composition are of a different type or subtype.

32. The method of claim 31, wherein the at least one influenza virus strain of the first immunogenic composition is of a A-type, and the at least one influenza virus strain of the second immunogenic composition is of a B-type.

33. The method of claim 32, wherein the second immunogenic composition further comprises a A-type influenza virus strain of a subtype different from, the A-type influenza virus strain included in the first immunogenic composition.

34. The method of claim 30, wherein the second immunogenic composition is administered one year after the first immunogenic composition.

35.-36. (canceled)
